(19) United States
(12) Patent Application Publication
(10) Pub. No.: US 2025/0059613 A1
(43) Pub. Date: Feb. 20, 2025

(54) METHOD AND REAGENT FOR TESTING NOVEL CORONAVIRUS

(71) Applicant: SHIMADZU CORPORATION, Kyoto (JP)

(72) Inventors: Shinichiro KOBAYASHI, Kyoto-shi, Kyoto (JP); Masamitsu SHIKATA, Kyoto-shi, Kyoto (JP); Kenji NINOMIYA, Kyoto-shi, Kyoto (JP); Naoko TAKAOKA, Kyoto-shi, Kyoto (JP); Hideaki MARUSE, Kyoto-shi, Kyoto (JP)

(73) Assignee: SHIMADZU CORPORATION, Kyoto (JP)

(21) Appl. No.: 17/912,379

(22) PCT Filed: Mar. 25, 2021
(86) PCT No.: PCT/JP2021/012663

§ 371 Date: Mar. 13, 2023

(30) Foreign Application Priority Data
Mar. 27, 2020 (WO) PCT/JP2020/014415
Oct. 2, 2020 (WO) PCT/JP2020/037492

(51) Int. Cl. C12Q 1/70; C12Q 1/6844

(52) U.S. Cl. CPC C12Q 1/701; C12Q 1/6844; C12Q 2600/166

(57) ABSTRACT

A method for testing a novel coronavirus, the method including steps of: mixing a specimen sample collected from a subject, or a mixed liquid of the specimen sample and a medium, with a specimen treatment liquid containing sodium hydroxide as a main component, to obtain a mixed liquid; incubating the mixed liquid; adding a master mix containing a reaction liquid, an internal standard substance, a primer, a probe, a reverse transcriptase, and a PCR enzyme to the mixed liquid after the incubation, to obtain a final mixed liquid; subjecting the final mixed liquid to a reverse transcription reaction treatment; and detecting, through use of the probe, DNA amplified by PCR using the DNA generated by the reverse transcription reaction treatment as a template.

### CROSS REFERENCE TO PRIOR APPLICATION

This application is a National Stage Patent Application of PCT International Patent Application No. PCT/JP2021/012663 (filed on Mar. 25, 2021) under 35 U.S.C. § 371, which is a continuation-in-part of PCT International Patent Application No. PCT/JP2020/037492 (filed on Oct. 2, 2020), which is a continuation-in-part of PCT International Patent Application No. PCT/JP2020/014415 (filed on Mar. 27, 2020), which are all hereby incorporated by reference in their entirety.

### REFERENCE TO AN ELECTRONIC SEQUENCE LISTING

The content of the ASCII text file of the sequence listing named “2023-03-13_1875-0014_SequenceListing.txt”, which is 3,536 bytes in size, was created on Mar. 8, 2023 and electronically submitted on Mar. 13, 2023, is herein incorporated by reference in its entirety.

### TECHNICAL FIELD

The present invention relates to a method for testing novel coronaviruses and a kit for carrying out the method.

### BACKGROUND ART

It is known that coronaviruses infect all animals such as livestock, laboratory animals, pets, wild animals, and the like, and cause various diseases. Among them, four types of cold syndrome and two types of severe pneumonia viruses infected from animals have been known as coronaviruses infecting humans.

Coronaviruses that cause colds are said to account for 10 to 15% of colds and 35% during the epidemic period. As these coronaviruses, HCoV-229E and HCoV-OC43 have been discovered in the 1960s, and two kinds of HCoV-NL63 and HCoV-HKU1 have been discovered in the 2000s.

On the other hand, horseshoe bats are believed to be a natural host for a SARS coronavirus discovered in 2002 (hereinafter, sometimes referred to as SARS-CoV). It is known that SARS-CoV causes severe acute respiratory syndrome and causes severe symptoms in the human body. In addition, it is known that a MERS coronavirus discovered in 2012 (hereinafter, sometimes referred to as MERS-CoV) originates from one-hump camels as an infection source, and causes severe pneumonia when infected with a human. Both coronaviruses have triggered worldwide epidemics, and caused many infected people.

Further, at the end of 2019, a novel coronavirus (hereinafter, sometimes referred to as SARS-CoV-2) has been newly found to cause acute respiratory illness. The first epidemic place is considered to be Wuhan City, Hubei Province, China, but the infection spread has become a worldwide epidemic, and infection has continued to expand from East Asia to Southeast Asia, Middle East, Europe, and America. The emergence of infected people in Brazil in early 2020 has spread the infection to all five continents excluding the Antarctic continent.

No effective prevention and treatment methods have been established for the novel coronavirus, and the novel coronavirus may cause severe pneumonia symptoms, and in the worst case, death, in some people, but the symptoms are not specific. For example, there are a wide range of symptoms from no symptoms to severe pneumonia to death. Typical symptoms are said to include fever, dry cough, fatigue, sputum, shortness of breath, sore throat, headache, muscle pain, joint pain, and the like.

Since the initial symptoms are similar to cold, it is difficult to distinguish them at an early stage of onset, and slight fever and cold symptoms may last for about one week after passing through the latent period from infection, but the initial symptoms of the patient are not limited to fever and cough specific to pneumonia, but may also be symptoms of the digestive system and the nervous system such as diarrhea, nausea, headache, general fatigue and the like, which are considered to make early diagnosis difficult.

Therefore, there is a need to establish a quick test method for determining whether or not infected with SARS-CoV-2. Among them, a PCR method using a polymerase chain reaction (hereinafter, sometimes referred to as PCR) is a method suitable for detecting a microorganism such as a virus because a very small amount of nucleic acid can be amplified and highly sensitive detection can be performed.

The National Institute of Infectious Diseases has published a method for detecting by a PCR method as a method for detecting SARS-CoV-2 (Non Patent Literature 1). In this method, virus-derived RNA is purified from a specimen containing SARS-CoV-2 which is an RNA virus, and the purified RNA is amplified as cDNA by a reverse transcription-polymerase chain reaction (hereinafter, sometimes referred to as a RT-PCR method), to detect SARS-CoV-2.

### CITATION LIST

### Non Patent Literature

Non Patent Literature 1: National Institute of Infectious Diseases Pathogen Detection Manual 2019-nCoV

### SUMMARY OF INVENTION

### Technical Problem

However, the SARS-CoV-2 detection method requires two hours or more for extraction and purification of RNA, thus, the purification takes time and effort, which is a problem in performing a multi-specimen treatment. In addition, since the test for detecting the SARS-CoV-2 requires a certain degree of skill, it is considered that it takes time to expand an institution for the test.

In the case of SARS-CoV-2, since it has been reported that a latent period before onset is over 1 week or more and that secondary infection is caused also during the latent period, a quick and highly accurate test method is desired. In addition, in view of worldwide epidemic of SARS-CoV-2, a test kit capable of easily and conveniently and highly-accurately testing SARS-CoV-2 is also desired.

Further, in order to prevent the spread of infection, it is important to avoid a false negative determination in which it is determined that the virus is not detected even though the SARS-CoV-2 is actually contained in the specimen in the test. However, if, for example, a substance that inhibits an enzymatic activity of reverse transcriptase and/or DNA polymerase is mixed in a virus preservation liquid and/or transport liquid constituting a specimen, a reaction of RT-PCR does not proceed, and even if SARS-CoV-2 is contained in the specimen, there is a possibility that false negative determination will be made. Therefore, a test kit capable of preventing false negative is desired. The term “negative” as used herein means below detection limit by a PCR method.

An object of the present invention is to provide a method for quickly and inexpensively testing the presence or absence of SARS-CoV-2 infection while preventing false negative, and a kit capable of easily and conveniently performing the method.

In the SARS-CoV-2 test, usually, a PCR master mix is added to a specimen treatment liquid obtained by extracting virus-derived RNA from a specimen, and RT-PCR is performed. The PCR master mix contains a reverse transcriptase, a DNA polymerase, a PCR primer, deoxynucleoside triphosphate (dNTP) as a substrate for a DNA polymerase for extending the PCR primer, and a probe for detecting a PCR amplification product. In the PCR master mix, there is a case where a non-specific amplification reaction between the primers occurs after preparation of the PCR master mix and before addition of RNA extracted from a specimen to the PCR master mix, and in such a case, it is considered that virus test accuracy is affected. In particular, in the SARS-CoV-2 detection step, the PCR master mix may be left for several hours after the preparation due to an irregular operation, and during this time, it is necessary to suppress unintended non-specific amplification reaction to maintain the test accuracy.

An object of the present invention is to provide a method for quickly and inexpensively testing the presence or absence of SARS-CoV-2 infection with high accuracy by suppressing the non-specific amplification reaction, and a kit capable of easily and conveniently performing the method.

### Solution to Problem

The present invention relates to
a method for testing a novel coronavirus, the method including steps of:
mixing a specimen sample collected from a subject, or a specimen mixed liquid of the specimen sample and a medium, with a specimen treatment liquid containing sodium hydroxide as a main component, to obtain a mixed liquid;
incubating the mixed liquid;
adding a master mix containing a reaction liquid, an internal standard substance, a primer, a probe, a reverse transcriptase, and a PCR enzyme to the mixed liquid after the incubation, to obtain a final mixed liquid;
subjecting the final mixed liquid to a reverse transcription reaction treatment; and
detecting, through use of the probe, DNA amplified by PCR using the DNA generated by the reverse transcription reaction treatment as a template.

The present invention further relates to
the method for testing a novel coronavirus, wherein the specimen treatment liquid or the reaction liquid contains deoxyadenosine triphosphate (dATP), deoxyguanosine triphosphate (dGTP), deoxycytidine triphosphate (dCTP), and deoxythymidine triphosphate (dTTP).

The present invention further relates to
a kit for testing a novel coronavirus, the kit including a specimen treatment liquid containing sodium hydroxide as a main component, a reaction liquid, an internal standard substance, a primer, a probe, a reverse transcriptase, and a PCR enzyme.

The present invention further relates to
the kit for testing a novel coronavirus, wherein the specimen treatment liquid or the reaction liquid contains dATP, dGTP, dCTP, and dTTP.

### Advantageous Effects of Invention

According to the present invention, it is possible to provide a method for quickly and inexpensively testing the presence or absence of SARS-CoV-2 infection while preventing generation of false negative without purifying viral RNA contained in a specimen, and a kit capable of easily and conveniently performing the method.

Usually, dNTP as a substrate for DNA polymerase is added to a reaction liquid for preparing a PCR master mix. However, according to the present invention, by adding dNTP to a specimen treatment liquid instead of the reaction liquid, a non-specific amplification reaction between PCR primers does not occur even after preparation of a PCR master mix, and by adding a mixed liquid of a specimen sample and a specimen treatment liquid to the PCR master mix, all amplification reactions are started. Therefore, the present invention makes it possible to test for the presence or absence of a novel coronavirus in a specimen sample with high accuracy.

### BRIEF DESCRIPTION OF DRAWINGS

FIG. 1 is a diagram showing an amplification curve in real-time RT-PCR when the content of a sample liquid containing artificial synthetic RNA obtained by synthesizing a part of SARS-CoV-2 RNA, a pharyngeal swab liquid, and a UTM medium in a mixed liquid (10 μL) for measurement by one-step RT-PCR is changed to 1, 3, 5, and 7 μL.

FIG. 2A is a diagram showing an amplification curve of real-time RT-PCR for a sample liquid without PCR reaction inhibition by a PCR reaction inhibitor in a sample liquid.

FIG. 2B is a diagram showing an amplification curve of real-time RT-PCR for a sample liquid accompanied by PCR reaction inhibition by a PCR reaction inhibitor in a sample liquid.

FIG. 3A is a diagram showing an amplification curve of a real-time RT-PCR reaction for a sample liquid determined to be positive.

FIG. 3B is a diagram showing an amplification curve of a real-time RT-PCR reaction for a sample liquid determined to be negative (below detection limit).

FIG. 4 is a diagram showing results of testing a nasal swab liquid and sputum (25 cases) as specimens by a method described in Pathogen Detection Manual 2019-nCoV (Non Patent Literature 1) and the method of the present invention.

FIG. 5 is a diagram showing results of testing saliva (22 cases) as a specimen by a method described in Pathogen Detection Manual 2019-nCoV (Non Patent Literature 1) and the method of the present invention.

### DESCRIPTION OF EMBODIMENTS

One of characteristics of a coronavirus is that its genome is not DNA but RNA. Therefore, when a PCR method is applied, a RT-PCR method is effective. The RT-PCR method includes a one-step RT-PCR method and a two-step RT-PCR method. The one-step RT-PCR method is preferable from a viewpoint that an operation is easy and convenient and contamination between samples is suppressed because a reverse transcription reaction and PCR are performed in the same container in succession.

The method for testing a novel coronavirus of the present invention includes a step of mixing a specimen sample collected from a subject for determining the presence or absence of infection or a mixed liquid of the specimen sample and a medium with a specimen treatment liquid containing sodium hydroxide as a main component. The specimen sample collected from a subject includes a pharyngeal swab liquid, a nasal swab liquid, sputum, a bronchial wash liquid, saliva, and the like. The medium contains a virus preservation liquid and the like.

The medium to be used provides a growth environment for a culture target in culture of microorganisms and biological tissues, and commercially available virus transportation/preservation media such as UTM medium (manufactured by Nippon Becton Dickinson Co., Ltd.), VTM (manufactured by Sugiyama-Gen Co., Ltd.), and the like can be suitably used. The specimen sample may be mixed with phosphate buffered saline (hereinafter, sometimes referred to as PBS) or the like in addition to the medium. In addition, the specimen sample collected from a subject may not need to be mixed with a medium.

The specimen treatment liquid is an aqueous solution containing sodium hydroxide as a main component, and is added for the purpose of extracting RNA from coronavirus particles. The specimen treatment liquid may contain, in addition to sodium hydroxide, a metal chelating agent such as glycol ether diamine tetraacetic acid (hereinafter, sometimes referred to as EGTA) or the like and/or a reducing agent such as dithiothreitol (hereinafter, sometimes referred to as DTT) or the like from the viewpoint of efficiently performing a RT-PCR treatment described later and enhancing the test accuracy.

The specimen sample or a mixed liquid of the specimen sample and a medium (hereinafter, sometimes both are collectively referred to as sample liquid) and the specimen treatment liquid are mixed, at a volume ratio of the specimen treatment liquid of 0.4 to 2.3 times with the volume of the sample liquid as 1.0, to obtain a mixed liquid. It is considered that by obtaining the mixed liquid mixed at the volume ratio, genomic RNA of coronavirus is appropriately extracted, and a reverse transcription reaction and PCR appropriately proceed. The mixing ratio of the sample liquid and the specimen treatment liquid is preferably 0.5 times or more, more preferably 0.8 to 1.3 times, and further preferably 0.9 to 1.1 times in terms of a volume ratio of the specimen treatment liquid, where the volume of the sample liquid is taken as 1.0.

It is preferable that the sample liquid and the specimen treatment liquid are mixed at the above-described mixing ratio, but the volume of a final mixed liquid to be described later is preferably about 25 μL or less from the viewpoint of being able to easily and conveniently cope with a small amount of the specimen sample and suppressing the used amount of expensive enzymes to reduce the test cost. When the volume of the final mixed liquid is 25 μL or less, it is preferable to obtain a mixed liquid by mixing 3 μL to 5 μL of the sample liquid and 3 μL to 5 μL of the specimen treatment liquid. The amount of each of the sample liquid and the specimen treatment liquid is more preferably 5 μL.

The obtained mixed liquid is incubated. The incubation temperature is appropriately set. From the viewpoint of the quickness of the test and the accuracy of the obtained result, the incubation temperature is normal temperature to 95° C., preferably 80 to 95° C., and the incubation time is preferably 3 minutes to 5 minutes. The normal temperature is usually around 25° C.

A master mix containing a reaction liquid, an internal standard substance, a PCR primer pair, a probe, a reverse transcriptase, and a PCR enzyme is added to the mixed liquid that has undergone the above incubation step, to obtain a final mixed liquid. The reaction liquid contains a PCR buffer containing a surfactant. The surfactant can be selected from anionic surfactants, cationic surfactants, amphoteric surfactants and nonionic surfactants.

The anionic surfactant includes, but is not limited to, alkyl sulfates, alkyl ether sulfates, docusates, sulfonate fluorosurfactants, alkyl benzene sulfonates, alkyl aryl ether phosphates, alkyl ether phosphates, alkyl carboxylates, sodium lauroyl sarcosinate, carboxylate fluorosurfactants, sodium cholate and sodium deoxycholate, and the like. As the alkyl sulfate, sodium dodecyl sulfate (SDS) and ammonium dodecyl sulfate are preferable, and sodium dodecyl sulfate is more preferable. Sodium dodecyl sulfate is also referred to as sodium lauryl sulfate (SLS). The cationic surfactant includes, but is not limited to, ethyltrimethylammonium bromide, hexadecyltrimethylammonium bromide, tetradecyltrimethylammonium bromide, and the like. The amphoteric surfactant includes, but is not limited to, betaines and alkylamino fatty acid salts, for example. The nonionic surfactant includes, but is not limited to, nonylphenoxypolyethoxyethanol (NP-40), polyoxyethylene sorbitan monooleate (Tween (registered trademark) 80), polyoxyethylene p-t-octylphenol (Triton X-100 (registered trademark)), and the like. The surfactant contained in the reaction liquid is preferably a nonionic surfactant, and in order to efficiently extract viral RNA, the concentration is preferably 0.05 to 5% (w/v).

The PCR buffer preferably contains KCl, MgCl₂, and a deoxyribonucleotide 5′-triphosphate (hereinafter, sometimes abbreviated as dNTP) mix from the viewpoint of performing efficient RT-PCR. The dNTP mix is an aqueous solution obtained by previously mixing deoxyadenosine triphosphate (hereinafter, sometimes abbreviated as dATP), deoxyguanosine triphosphate (hereinafter, sometimes abbreviated as dGTP), deoxycytidine triphosphate (hereinafter, sometimes abbreviated as dCTP), and deoxythymidine triphosphate (hereinafter, sometimes abbreviated as dTTP) at a predetermined concentration. In addition, the PCR buffer is not particularly limited, and examples of the PCR buffer include a phosphate buffer, a trishydroxymethylaminomethane (Tris) buffer, a borate buffer, and a Good buffer such as HEPES or the like, but a Tris-HCl buffer is preferable from the viewpoint of performing efficient RT-PCR. The concentrations of dNTP, MgCl₂, KCl, and the buffer can be appropriately set according to a RT-PCR treatment described later. For example, concentrations of 1.5 mM for MgCl₂, 35 mM for KCl, 200 μM for each dNTP, and 10 mM for Tris can be exemplified.

The dNTP mix may be contained in the specimen treatment liquid containing sodium hydroxide as a main component instead of the PCR buffer constituting the reaction liquid. In this case, since the master mix does not contain dNTPs (dATP, dGTP, dCTP and dTTP), it is possible to suppress a non-specific amplification reaction between primers that may occur after preparation of the master mix.

Any 1 to 3 dNTPs selected from dATP, dGTP, dCTP, and dTTP may be added to the PCR buffer, and the remaining dNTPs may be contained in the specimen treatment liquid. Preferably, any two dNTPs selected from dATP, dGTP, dCTP, and dTTP are added to the PCR buffer, and the remaining two dNTPs are added to the specimen treatment liquid. As described above, even when the master mix contains any 1 to 3 dNTPs selected from dATP, dGTP, dCTP, and dTTP, it is possible to suppress a non-specific amplification reaction between primers that may occur after preparation of the master mix.

In order to prevent carryover of a PCR product from the PCR, a dNTP mix in which dTTP is replaced with dUTP (deoxyuridine triphosphate) may be contained in the specimen treatment liquid. Since the amplification product incorporating deoxyuridine (dU) can be decomposed by uracil-N-glycosylase (UNG) treatment, the amplification product containing dU mixed in the PCR reaction liquid can be decomposed by UNG before PCR, and false negatives due to the influence of the amplification product of the PCR can be prevented.

In a specimen sample collected from a subject, a biological origin negative charge substance (for example, certain kinds of sugars, dyes, and the like) to be adsorbed to DNA polymerase and a biological origin positive charge substance (for example, certain kinds of proteins and the like) to be adsorbed to DNA may be mixed. These negative charge substances and positive charge substances inhibit PCR, thus making accurate measurements difficult. In order to cope with this problem of PCR inhibition, a substance that binds to these negative charge substances and positive charge substances to neutralize the PCR inhibitory action by these charge substances is added to the PCR buffer. As the PCR buffer, a reagent for gene amplification Ampdirect (registered trademark, Shimadzu Corporation) or Ampdirect Plus (registered trademark, Shimadzu Corporation) can be used. The use of such a reagent for gene amplification is preferable from the viewpoint that PCR or the like can be performed with a smaller amount of sample because the treatment for purifying nucleic acids, such as solid phase extraction, liquid-liquid extraction, and the like becomes unnecessary, and a liquid does not need to be discarded.

In the present invention, the internal standard substance includes at least one of the following (1) and (2). (1) An internal standard nucleic acid and (2) a set of primer pairs and probes required for amplification of internal standard nucleic acids by PCR. In the present invention, the internal standard nucleic acid is a sequence that does not cause a cross-reaction with primers and probes for detecting SARS-CoV-2 in PCR. The internal standard nucleic acid is a nucleic acid that has a sequence different from that of a SARS-CoV-2 RNA-derived nucleic acid and is amplified independently of the virus-derived nucleic acid, and may be either DNA or RNA. The internal standard nucleic acid may be added to a master mix for performing RT-PCR, or may be a nucleic acid derived from a specimen. In addition, in order to improve an amplification efficiency, a chain length of the internal standard nucleic acid is preferably 300 bp or less, and more preferably 100 bp or less. For example, when the internal standard nucleic acid is DNA, the internal standard DNA is DNA that has a sequence different from that of cDNA generated from SARS-CoV-2 RNA by a reverse transcription reaction in PCR and is amplified independently of the virus-derived cDNA. That is, the internal standard nucleic acid can be used as an index for determining whether PCR has appropriately progressed. In PCR, when the internal standard nucleic acid is amplified, it is indicated that the PCR has appropriately progressed, but when the internal standard nucleic acid is not amplified, it is indicated that the PCR itself has not progressed. Therefore, it is possible to avoid an erroneous determination (false negative) that a SARS-CoV-2 gene is not detected even though SARS-CoV-2 is contained in the specimen sample. An example of the internal standard nucleic acid may be a nucleic acid having an artificially designed sequence, a sequence derived from another organism, or a nucleic acid derived from a specimen as long as the internal standard nucleic acid does not affect the amplification of the SARS-CoV-2 gene. When such an internal standard nucleic acid is added to a master mix, a forward primer and a reverse primer for amplification by PCR of a nucleic acid that does not affect the amplification of the SARS-CoV-2 gene, and a probe for detecting an amplification product by the primer pair are contained in the kit of the present invention. When a nucleic acid derived from a specimen is used as the internal standard nucleic acid, a forward primer and a reverse primer for amplification of a nucleic acid derived from a specimen that does not affect the amplification of the SARS-CoV-2 gene, and a probe for detecting an amplification product by the primer pair are contained in the kit of the present invention.

In the present invention, for PCR primer pairs (forward and reverse) for amplifying a target gene, primers specific to a sequence of a nucleic acid derived from SARS-CoV-2 RNA can be used, and for example, primers specific to a sequence of cDNA generated from SARS-CoV-2 RNA by a reverse transcription reaction can be used. Examples of the primer include primer pairs described in Table 1 and primer pairs described in Table 2. Detection targets of the exemplified PCR primer pairs are two regions of N (nucleocapsid) gene. The method of National Institute of Infectious Diseases includes N set N_Sarbeco_F1 (Forward, SEQ ID NO: 1) and N_Sarbeco_R1 (Reverse, SEQ ID NO: 2) and N set No. 2 NIID_2019-nCoV_N_F2 (Forward, SEQ ID NO: 3) and NIID_2019-nCoV_N_R2 (Reverse, SEQ ID NO: 4), and the method of U.S. Centers for Disease Control and Prevention includes N1 Forward Primer (SEQ ID NO: 5) and N1 Reverse Primer (SEQ ID NO: 6), and N2 Forward Primer (SEQ ID NO: 7) and N2 Reverse Primer (SEQ ID NO: 8), as examples. The PCR primer pair for amplifying the internal standard nucleic acid is preferably a PCR primer pair that hybridizes to the internal standard nucleic acid under stringent conditions and does not hybridize to the SARS-CoV-2 derived nucleic acid. The stringent condition refers to a condition in which binding between a template nucleic acid and a primer is specific in annealing in PCR, which is a step in which the primer binds to the template nucleic acid.

TABLE 1

| | | |
|---|---|---|
| | | Seq. |
| Name | Sequence (5′ to 3′) | ID No. |

N set

| | | |
|---|---|---|
| (1) N_Sarbeco_F1 | CACATTGGCACCCGCAATC | 1 |
| (2) N_Sarbeco_R1 | GAGGAACGAGAAGAGGCTTG | 2 |
| (3) N_Sarbeco_P1 | FAM-ACTTCCTCAAGGAACA | 9 |
| | ACATTGCCA-BHQ1 | |

N set No. 2

| | | |
|---|---|---|
| (4) NIID_2019- | AAATTTTGGGGACCAGGAAC | 3 |
| nCoV_N_F2 | | |
| (5) NIID_2019- | TGGCAGCTGTGTAGGTCAAC | 4 |
| nCoV_N_R2 | | |
| (6) NIID_2019- | ROX-ATGTCGCGCATTGGCA | 10 |
| nCoV_N_P2 | | |
| | TGGA-BHQ2 | |

TABLE 2

| | | |
|---|---|---|
| Name | Sequence (5′ to 3′) | Seq. ID No. |

2019-nCoV_N1 (N1 set)

| | | |
|---|---|---|
| N1 Forward | GACCCCAAAAT | 5 |
| Primer | CAGCGAAAT | |
| N1 Reverse | TCTGGTTACTGCCAGTTG | 6 |
| Primer | AATCTG | |
| N1 Probe | ROX-ACCCCGCATTACGT | 11 |
| | TTGGTGGACC-BHQ2 | |

2019-nCoV_N2 (N2 set)

| | | |
|---|---|---|
| N2 Forward | TTACAAACATTG | 7 |
| Primer | GCCGCAAA | |
| N2 Reverse | GCGCGACATTCC | 8 |
| Primer | GAAGAA | |
| N2 Probe | FAM-ACAATTTGCCCCCA | 12 |
| | GCGCTTCAG-BHQ1 | |

From the viewpoint of test quickness, in RT-PCR described later, a PCR product is monitored by real-time measurement. When the real-time measurement is performed, RT-PCR and a step of detecting the RT-PCR product are performed in the same container. The real-time measurement of the PCR product is also referred to as real-time PCR. In the real-time PCR, usually, a PCR amplification product is detected by fluorescence. Examples of the fluorescence detection method include a method using an intercalating fluorescent dye and a method using a fluorescently labeled probe. As the intercalating fluorescent dye, for example, SYBR (registered trademark) Green I is used. The intercalating fluorescent dye binds to double-stranded DNA synthesized by PCR and emits fluorescence by irradiation with excitation light. By measuring the fluorescence intensity, the production amount of the PCR amplification product can be measured.

In order to fluorescently detect a PCR amplification product, a probe labeled with one or more fluorescent dyes is added in the method for testing a novel coronavirus of the present invention. Examples of the probe include a hydrolysis probe, Molecular Beacon, and the like. The hydrolysis probe is an oligonucleotide in which the 5′ end is modified with a fluorescent dye and the 3′ end is modified with a quencher substance. The hydrolysis probe specifically hybridizes to a template DNA in an annealing step of PCR, but a quencher is present on the hydrolysis probe, therefore, generation of fluorescence is suppressed even if irradiated with excitation light. In the subsequent extension reaction step, for example, when the hydrolysis probe hybridized to the template DNA is decomposed by the 5′→3′ exonuclease activity of Taq DNA polymerase, the fluorescent dye is released from the hydrolysis probe, and the suppression of generation of fluorescence by the quencher is released to emit fluorescence. By measuring the fluorescence intensity, the production amount of the amplification product can be measured.

As examples of the fluorescent dye, FAM (6-carboxyfluorescein), ROX (6-carboxy-X-rhodamine), Cy3 and Cy5 (Cyanine-based dyes), HEX (4,7,2′,4′,5′,7′-hexachloro-6-carboxyfluorescein), and the like can be used as fluorescence to label a probe. Examples of the quencher include TAMRA (registered trademark), Black Hole Quencher (BHQ, registered trademark)1, BHQ2, MGB-Eclipse (registered trademark), DABCYL, and the like.

In the present invention, examples of the oligonucleotide fluorescently labeled probe that can be used for detecting a PCR amplification product of cDNA derived from SARS-CoV-2 RNA include probes described in Table 1 or Table 2. As probes that hybridize under stringent conditions to genes amplified by PCR primer pairs of N set and N set No. 2 described in Table 1, N_Sarbeco_P1 and NIID_2019-nCoV_N_P2 are shown, respectively. For the N_Sarbeco_P1, FAM as a fluorescent dye at the 5′ end and BHQ1 as a quencher substance at the 3′ end are bound. For the NIID_2019-nCoV_N_P2, ROX as a fluorescent dye at the 5′ end and BHQ2 as a quencher substance at the 3′ end are bound. As probes that hybridize under stringent conditions to genes amplified by N1 set and N2 set PCR primer pairs described in Table 2, N1 Probe and N2 Probe are shown, respectively. For the N1 Probe, ROX as a fluorescent dye at the 5′ end and BHQ2 as a quencher substance at the 3′ end are bound. For the N2 Probe, FAM as a fluorescent dye at the 5′ end and BHQ1 as a quencher substance at the 3′ end are bound. In the fluorescent dye at the 5′ end of the probe, FAM may be replaced with ROX, and ROX may be replaced with FAM. Although further other fluorescent dyes can be used, probes that hybridize to two regions of an amplified N gene of SARS-CoV-2 bind mutually different fluorescent dyes.

In order to detect the PCR amplification product of the internal standard nucleic acid, preferable is an oligonucleotide fluorescently labeled probe that hybridizes to the internal standard nucleic acid under stringent conditions and to which a fluorescent dye different from a fluorescent dye that binds to a fluorescent probe for detecting SARS-CoV-2 binds. When two kinds of fluorescent probes for detecting SARS-CoV-2 described in Table 1 are used, Cy5 is exemplified as a fluorescent dye of a fluorescent probe for internal standard nucleic acid detection, but the fluorescent dye is not limited to this. As described above, in the present invention, in the oligonucleotide fluorescently labeled probe used for detection of a PCR amplification product, fluorescent dyes that bind to respective probes are different from each other, so that PCR amplification products by a plurality of primer pairs can be separated and detected.

The reverse transcriptase is an enzyme that generates single-stranded complementary DNA (cDNA) using coronavirus RNA as a template. For example, RNA virus-derived RNA-dependent DNA polymerases such as avian myeloblastosis virus (AMV), moloney murine leukemia virus (M-MLV), human immunodeficiency virus (HIV) and the like, and variants of them, can be used. The reverse transcriptase is preferably an enzyme having an activity of 200 U or more.

The DNA polymerase that is a PCR enzyme is preferably a heat-resistant DNA polymerase derived from thermophilic bacteria, and examples of the DNA polymerase include Taq, Tth, KOD, Pfu, and variants of them. From the viewpoint of avoiding non-specific amplification by the DNA polymerase, a hot-start DNA polymerase may be used. Examples of the hot-start DNA polymerase include a DNA polymerase to which an anti-DNA polymerase antibody is bound or a DNA polymerase obtained by heat-sensitive chemical modification of an enzyme active site, with the DNA polymerase to which an anti-DNA polymerase antibody is bound being preferable. The PCR enzyme is preferably an enzyme having an activity of 3 U or more.

A master mix containing a reaction liquid, an internal standard substance, a PCR primer pair, a probe, a reverse transcriptase and a PCR enzyme is added to the incubated mixed liquid, to obtain a final mixed liquid. When the volume of the final mixed liquid is about 25 μL or less, it is preferable to add the master mix in a range of 14 to 16 μL.

The resulting final mixed liquid is subjected to RT-PCR treatment. The reaction temperature condition of the reverse transcription reaction in RT-PCR and the PCR conditions (temperature, time and number of cycles) are appropriately set. In addition, RT-PCR is performed in a commercially available reaction tube for RT-PCR.

By detecting RNA amplified by the RT-PCR treatment using the labeled fluorescent-labeled probe, the presence or absence of SARS-CoV-2 can be determined in real time by real-time measurement, and a quick test of a novel coronavirus can be performed.

In real-time measurement of a PCR product, the progress of PCR can be confirmed in real time by monitoring an amplification curve of the PCR product using a fluorescent filter corresponding to a fluorescent dye to be used. When the fluorescence intensity increases according to the number of PCR cycles, it is determined that the presence of SARS-CoV-2 in a specimen sample is positive, whereas when the fluorescence intensity does not increase in PCR, it is determined to be negative. At this time, when the internal standard substance is added to the master mix, if the fluorescence intensity corresponding to the internal standard nucleic acid increases according to the number of PCR cycles, possibility of false negative can be easily eliminated. As an example of the internal standard nucleic acid, any internal standard nucleic acid that does not affect the amplification of SARS-CoV-2 may be used, and the internal standard nucleic acid may have a sequence derived from another organism, an artificially designed sequence, or a sequence derived from a specimen.

In order to efficiently perform the above method, the present invention further provides a kit for testing a novel coronavirus having a specimen treatment liquid containing sodium hydroxide, a reaction liquid, an internal standard substance, a PCR primer pair, a probe, a reverse transcriptase, and a PCR enzyme. The test kit makes it possible to efficiently perform a test when a very small amount of specimen is collected and the test for the novel coronavirus is performed according to each of the steps described above. The specimen treatment liquid, the reaction liquid, the internal standard substance, the PCR primer pair, the probe, the reverse transcriptase, and the PCR enzyme are as described above.

Preferably, the PCR primer pair contains one or more PCR primer pairs for amplifying nucleic acids derived from SARS-CoV-2 RNA and a PCR primer pair for amplifying an internal standard nucleic acid. When two or more PCR primer pairs for amplifying SARS-CoV-2 gene are contained, it is preferable to select primer pairs each hybridizing to a different DNA sequence in order to improve virus detection accuracy.

In the oligonucleotide fluorescently labeled probe, fluorescent dyes that bind to the respective probes are different from each other, therefore, a kit capable of individually measuring a PCR amplification product is provided.

In the test kit, a specimen treatment liquid, a reaction liquid, an internal standard substance, a PCR primer pair, a probe, a reverse transcriptase, and a PCR enzyme may be contained in different containers, however, according to the procedure of the method for testing a novel coronavirus of the present invention, it is preferable to mix them in advance in a predetermined amount as appropriate, because it is possible to avoid complication of mixing at the time of testing.

For example, a specimen treatment liquid may be contained in one container, and a reaction liquid, an internal standard substance, a PCR primer pair, a probe, a reverse transcriptase, and a PCR enzyme may be mixed in predetermined amounts and contained in one container. In addition, a reaction liquid, an internal standard substance, a PCR primer pair, and a probe may be mixed in predetermined amounts and contained in one container, and a reverse transcriptase and a PCR enzyme may be mixed in predetermined amounts and contained in another container.

From the viewpoint of complexity of mixing at the time of test and preservation stability, it is preferable to distribute these in two to four containers, and for example, a specimen treatment liquid, a reaction liquid and an internal standard substance, and a PCR primer pair and a probe mixed in predetermined amounts, and a reverse transcriptase and a PCR enzyme mixed in predetermined amounts, may be contained independently in separate containers.

By using the method and kit for testing a novel coronavirus of the present invention, it is possible to quickly and easily and conveniently test for the presence or absence of SARS-CoV-2 infection while preventing generation of false negative. Since the RT-PCR test has higher detection sensitivity than immunochromatography, it is possible to provide a coronavirus test capable of accurately determining in a short time even in a case of a subject infected with SARS-CoV-2, although the subject is asymptomatic for high fever, cough, and the like.

### EXAMPLES

Next, the present invention will be described in detail with reference to examples, but the scope of the present invention is not limited to them.

### First Example

### Preparation of Mixed Liquid

The following RT-PCR was performed using artificial synthetic RNA (10,000 copies) obtained by synthesizing a part of genomic RNA of a novel coronavirus. In a PCR tube, 9, 7, 5 or 3 μL of a specimen treatment liquid containing sodium hydroxide was added to 1, 3, 5 or 7 μL of a sample liquid containing a UTM medium (manufactured by Nippon Becton Dickinson Co., Ltd.), the artificial synthetic RNA and a pharyngeal swab liquid, respectively, to make a total amount 10 μL.

The obtained mixed liquid was mixed with a vortex mixer, incubated at 90° C. for 5 minutes in a thermostatic apparatus, and then ice-cooled.

Reagent A (6.5 μL), reagent B (6.5 μL), and reagent C (2 μL) were mixed with a vortex mixer to obtain a master mix. The compositions of reagent A, reagent B, and reagent C are as follows.
Reagent A: Reaction liquid containing surfactant and PCR buffer
Reagent B: PCR primer pair and probe
Reagent C: 250 U reverse transcriptase and 3.125 U PCR enzyme

The master mix (15 μL) was added to the PCR tube containing 10 μL of the mixed liquid after the incubation. Thereafter, the mixture was mixed with a vortex mixer, the PCR tube was set in a real-time PCR apparatus, and PCR was immediately started.

Real-time RT-PCR setting conditions are as follows.

### RT-PCR Setting Conditions

After holding at 50° C. for 10 minutes, hold at 95° C. for 1 minute. Thereafter, 45 cycles of a cycle including holding at 95° C. for 5 seconds, then lowering the temperature to 60° C., and holding for 30 seconds were performed.

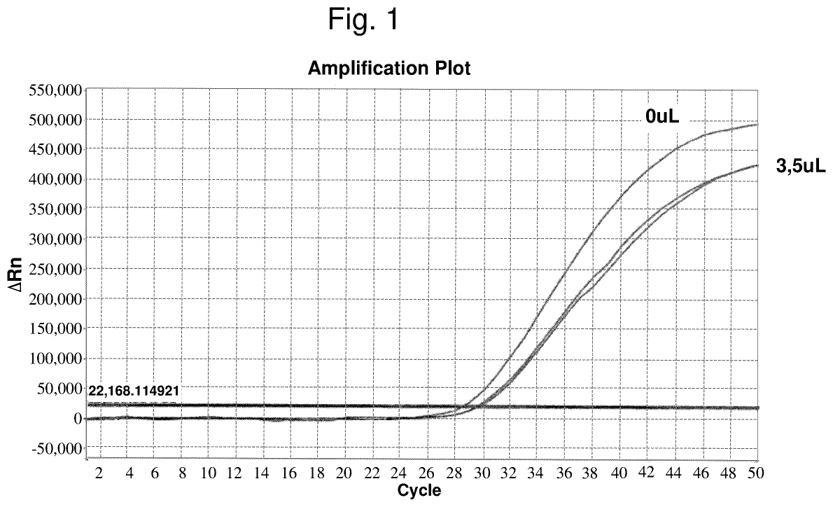

Results of plotting the amplification curves of real-time RT-PCR against the number of cycles are shown in FIG. 1. When the amount of the sample liquid was 3 and 5 μL, there was not a much difference in the rising of the amplification curve as compared with a case where no medium was added.

From the above results, in the SARS-CoV-2 test, a sample containing a medium may be handled, but when a sample containing a medium is directly added to a reaction liquid, PCR is considered to be affected. The method for testing a novel coronavirus of the present invention eliminates such an influence and can quickly obtain a result of the presence or absence of the novel coronavirus.

### Second Example

### Confirmation of False Negative in Viral Gene Detection

A pharyngeal swab liquid was obtained as a specimen sample from a plurality of subjects, and a UTM medium was mixed for each specimen sample to prepare a plurality of sample liquids. A specimen treatment liquid (5 μL) containing sodium hydroxide was added to 5 μL of each sample liquid, and the mixture was mixed with a vortex mixer. The resulting mixed liquid was incubated at 90° C. for 5 minutes in a thermostatic apparatus, and then ice-cooled.

Reagent A (6.5 μL), reagent B (6.5 μL), and reagent C (2 μL) were mixed with a vortex mixer to obtain a master mix. The compositions of reagent A, reagent B, and reagent C are as follows.
Reagent A: Reaction liquid containing internal standard DNA (76 bp), surfactant and PCR buffer
Reagent B: PCR primer pair and probe
Reagent C: 250 U reverse transcriptase and 3.125 U PCR enzyme

To a PCR tube containing 10 μL of the mixed liquid after the incubation, 15 μL of the master mix containing two PCR primer pairs having different gene amplification regions and probes (N1 set and N2 set described in Table 2) and a PCR primer pair for internal standard DNA detection and a Cy5-labeled probe was added. Thereafter, the mixture was mixed with a vortex mixer, the PCR tube was set in a real-time PCR apparatus, and PCR was immediately started. RT-PCR was performed under the same conditions as in First Example.

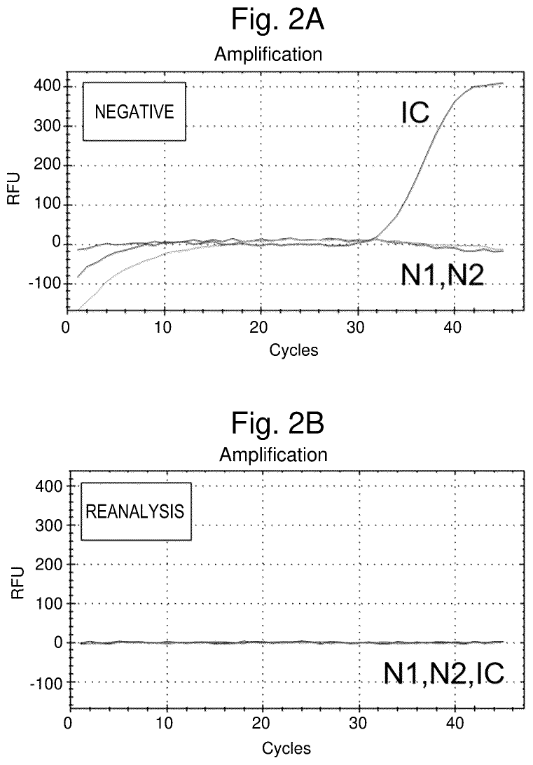

An amplification curve of real-time RT-PCR for a sample liquid without PCR reaction inhibition by a PCR reaction inhibitor in the sample liquid, among the plurality of sample liquids prepared as described above, is shown in FIG. 2A. Amplification of N1 and N2 regions of the SARS-CoV-2 gene is not observed, but a rising of the amplification curve of internal standard DNA (IC) is observed, indicating that PCR has progressed properly. As a result, it is determined that the sample liquid is truly negative, not containing a viral gene. An amplification curve of real-time RT-PCR for a sample liquid accompanied by PCR reaction inhibition by a PCR inhibitor in the sample liquid, among the plurality of sample liquids described above, is shown in FIG. 2B. Since a rising of the amplification curve of the internal standard DNA (IC) is not observed, it is not possible to distinguish whether it is negative (below detection limit) or false negative. In such a case, generation of false negative can be prevented by performing reanalysis.

### Third Example

### PCR Amplification Curve Determined as SARS-CoV-2 Positive or Negative

Using a sample liquid to which the SARS-CoV-2 gene was added or not added, an amplification curve in the case of being determined to be virus positive or negative in real-time RT-PCR was examined. A specimen treatment liquid (5 μL) containing sodium hydroxide was added to 5 μL of a sample liquid containing artificial synthetic RNA (10,000 copies) obtained by synthesizing a part of genomic RNA of the SARS-CoV-2 gene, and the mixture was mixed with a vortex mixer. The resulting mixed liquid was incubated at 90° C. for 5 minutes in a thermostatic apparatus, and then ice-cooled.

Reagent A (6.5 μL), reagent B (6.5 μL), and reagent C (2 μL) were mixed with a vortex mixer to obtain a master mix. The compositions of reagent A, reagent B, and reagent C are as follows.
Reagent A: Reaction liquid containing internal standard DNA (76 bp), surfactant and PCR buffer
Reagent B: PCR primer pair and probe
Reagent C: 250 U reverse transcriptase and 3.125 U PCR enzyme

To a PCR tube containing 10 μL of the mixed liquid after the incubation, 15 μL of the master mix containing two PCR primer pairs having different gene amplification regions and probes (N1 set and N2 set described in Table 1) and a PCR primer pair for internal standard DNA detection and a Cy5-labeled probe was added. Thereafter, the mixture was mixed with a vortex mixer, the PCR tube was set in a real-time PCR apparatus, and PCR was immediately started. RT-PCR was performed under the same conditions as in First Example. For the measurement, a CFX 96 Touch Deep Well real-time PCR analysis system (Bio-Rad) was used, and Cq value (the number of cycles at which an amplification curve intersects a threshold line) analysis setting was as follows.
Cq Determination Mode: Single Threshold
Baseline Setting: Baseline Subtracted Curve Fit
Here, a Cq value≤40 was determined to be positive.

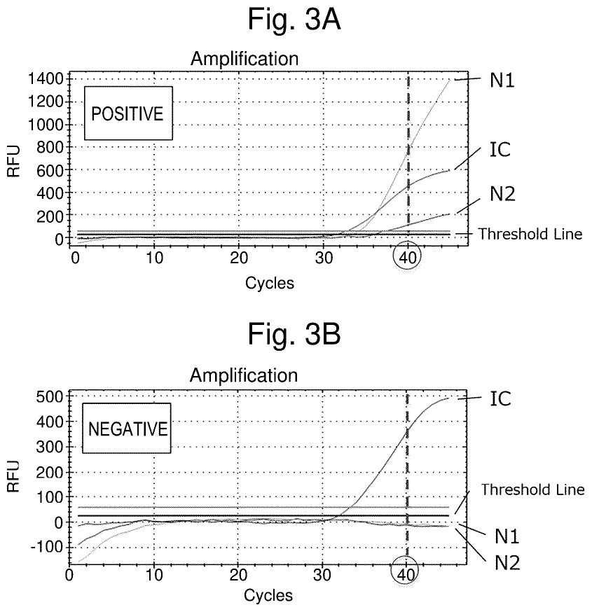

FIG. 3A shows an amplification curve when RT-PCR is performed on a sample liquid containing a SARS-CoV-2 gene. In this case, a rising of the amplification curve of N1 and N2 regions of the SARS-CoV-2 gene and internal standard DNA (IC) are observed, indicating that PCR has progressed properly. As a result, it is determined that the sample liquid is positive, containing a viral gene.

FIG. 3B shows an amplification curve when RT-PCR is performed on a sample liquid containing no SARS-CoV-2 gene. In this case, although amplification of N1 and N2 regions of the SARS-CoV-2 gene is not observed, a rising of the amplification curve of the internal standard DNA (IC) is observed, which indicates that PCR has appropriately progressed. As a result, it is determined that the sample liquid is negative (below detection limit), containing no viral gene.

### Fourth Example

### Comparison (1) of Method of Present Invention With Method Described in Non Patent Literature “Pathogen Detection Manual 2019-nCoV”, in Determination of SARS-CoV-2 Infection

Clinical specimens (nasal swab liquid and sputum) (25 cases) mixed with a UTM medium were measured by the method of the present invention and a method described in Non Patent Literature “Pathogen Detection Manual 2019-nCoV”, and the determination results were compared. The measurement by the method of the present invention was performed under the same conditions as in Third Example. The dNTP mix was added to a specimen treatment liquid containing sodium hydroxide in advance.

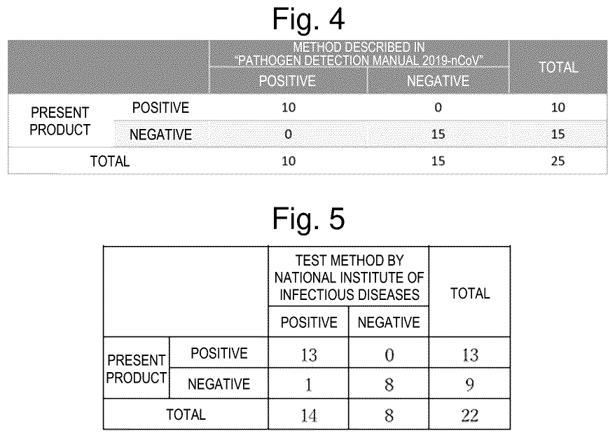

In the method of the present invention, a Cq value≤40 was determined to be positive, and the N1 region and/or the N2 region of the viral gene was detected. As a result, in the method of the present invention, 10 cases were positive and 15 cases were negative, and all cases were consistent with the method described in “Pathogen Detection Manual 2019-nCoV” (FIG. 4). Thus, it has been shown that the method of the present invention provides highly reliable test results.

### Fifth Example

### Comparison (2) of Method of Present Invention With Method Described in Non Patent Literature “Pathogen Detection Manual 2019-nCoV”, in Determination of SARS-CoV-2 Infection

Clinical specimens (saliva) (22 cases) not mixed with a medium were measured by the method of the present invention and a method described in Non Patent Literature “Pathogen Detection Manual 2019-nCoV”, and the test results were compared. The measurement by the method of the present invention was performed under the same conditions as in Third Example. The dNTP mix was added to a specimen treatment liquid containing sodium hydroxide in advance.

In the method of the present invention, a Cq value≤40 was determined to be positive, and the N1 region and/or the N2 region of the viral gene was detected. As a result, in both methods, the positive matching rate was 93% (13/14), the negative matching rate was 100% (8/8), and the overall matching rate was 95% (21/22) (FIG. 5). As described above, it was shown that the method of the present invention gives a highly reliable test result even when saliva not mixed with a medium is used as a specimen sample.

In Second Example and Third Example, an example in which an internal standard DNA is added as an internal standard nucleic acid to a master mix has been shown, but instead of the internal standard DNA, a nucleic acid sequence that is derived from a specimen sample and does not affect the amplification of a SARS-CoV-2 gene can be used. That is, in Second Example and Third Example, an internal standard DNA is not added to reagent A, and in reagent B, a primer pair and a probe for amplification of a nucleic acid sequence derived from a specimen sample that does not affect the amplification of a SARS-CoV-2 gene are added, as a result, the same effect as in Second Example and Third Example can be obtained using a nucleic acid sequence derived from a specimen sample as an internal standard.

The dNTPs may be contained in the master mix by being added to a reaction liquid (reagent A above) containing a PCR buffer, but in Fourth Example and Fifth Example, dNTPs were added to the specimen treatment liquid to be added to a specimen sample. Owing to the addition of dNTPs to the specimen treatment liquid, it is possible to suppress a non-specific amplification reaction due to primers, which may occur until the master mix is added to the specimen treatment liquid containing a specimen sample to start the amplification of a SARS-CoV-2 gene, after the preparation of the master mix. The dNTPs are hardly decomposed during a viral RNA extraction operation by heat-treating a mixture of a specimen sample and a specimen treatment liquid at 90° C. for 5 minutes.

### Modes

It will be understood by those skilled in the art that the exemplary embodiments described above are specific examples of the following modes.

[1] A method for testing a novel coronavirus, the method including steps of:
mixing a specimen sample collected from a subject, or a specimen mixed liquid of the specimen sample and a medium, with a specimen treatment liquid containing sodium hydroxide as a main component, to obtain a mixed liquid;
incubating the mixed liquid;
adding a master mix containing a reaction liquid, an internal standard substance, a primer, a probe, a reverse transcriptase, and a PCR enzyme to the mixed liquid after the incubation, to obtain a final mixed liquid;
subjecting the final mixed liquid to a reverse transcription reaction treatment; and
detecting, through use of the probe, DNA amplified by PCR using the DNA generated by the reverse transcription reaction treatment as a template.

According to the invention of the above [1], it is possible to provide a method for quickly and inexpensively testing the presence or absence of SARS-CoV-2 infection. In addition, since the RT-PCR test has higher detection sensitivity than immunochromatography, it is possible to provide a highly sensitive coronavirus test in a short time even for a subject who is infected but does not have symptoms such as high fever, cough, and the like.

[2] The method for testing a novel coronavirus described in the above [1], wherein an amount of the master mix is 14 μL to 16 μL.

[3] The method for testing a novel coronavirus described in the above [1] or [2], wherein an amount of the final mixed liquid is 24 μL to 26 μL.

[4] The method for testing a novel coronavirus described in any one of the above [1] to [3], wherein the incubation is performed at a temperature from normal temperature to 95° C. for 3 minutes to 5 minutes.

[5] The method for testing a novel coronavirus described in any one of the above [1] to [4], wherein the specimen treatment liquid further contains at least one of glycol ether diamine tetraacetic acid and dithiothreitol.

[6] The method for testing a novel coronavirus described in any one of the above [1] to [5], wherein the specimen treatment liquid or the reaction liquid contains dATP, dGTP, dCTP, and dTTP.

[7] The method for testing a novel coronavirus described in any one of the above [1] to [5], wherein the specimen treatment liquid contains dATP, dGTP, dCTP, and dUTP.

[8] A kit for testing a novel coronavirus, the kit including a specimen treatment liquid containing sodium hydroxide as a main component, a reaction liquid, an internal standard substance, a primer, a probe, a reverse transcriptase, and a PCR enzyme.

[9] The kit for testing a novel coronavirus described in the above [8], containing two to four containers.

[10] The kit for testing a novel coronavirus described in the above [9], wherein the specimen treatment liquid, the reaction liquid and the internal standard substance, the primer and the probe, and the reverse transcriptase and the PCR enzyme are each independently contained in four containers.

[11] The kit for testing a novel coronavirus described in any one of [8] to [10], wherein the specimen treatment liquid or the reaction liquid contains dATP, dGTP, dCTP and dTTP.

[12] The kit for testing a novel coronavirus described in any one of [8] to [10], wherein the specimen treatment liquid contains dATP, dGTP, dCTP, and dUTP.

According to the inventions of the above [8] to [12], it is possible to quickly perform the method for testing the presence or absence of SARS-CoV-2 infection. In addition, since the RT-PCR test has higher detection sensitivity than immunochromatography, it is possible to perform a highly sensitive coronavirus test in a short time even for a subject who is infected but does not have symptoms such as high fever, cough and the like.

1. A method for testing a novel coronavirus, the method comprising steps of:
mixing a specimen sample collected from a subject, and/or a specimen mixed liquid of the specimen sample and a medium, with a specimen treatment liquid containing sodium hydroxide as a main component, to obtain a mixed liquid;
incubating the mixed liquid;
adding a master mix containing a reaction liquid, an internal standard substance, a primer, a probe, a reverse transcriptase, and a PCR enzyme to the mixed liquid after the incubation, to obtain a final mixed liquid;
subjecting the final mixed liquid to a reverse transcription reaction treatment; and
detecting, through use of the probe, DNA amplified by PCR using the DNA generated by the reverse transcription reaction treatment as a template
wherein an amount of the master mix is 14 μL to 16 μL.

2. (canceled)

3. The method for testing a novel coronavirus according to claim 1, wherein an amount of the final mixed liquid is 24 μL to 26 μL.

4. The method for testing a novel coronavirus according to claim 1, wherein the incubation is performed at a temperature from normal temperature to 95° C. for 3 minutes to 5 minutes.

5. The method for testing a novel coronavirus according to claim 1, wherein the specimen treatment liquid further contains at least one of glycol ether diamine tetraacetic acid and dithiothreitol.

6. The method for testing a novel coronavirus according to claim 1, wherein the specimen treatment liquid or the reaction liquid contains dATP, dGTP, dCTP, and dTTP.

7. The method for testing a novel coronavirus according to claim 1, wherein the specimen treatment liquid contains dATP, dGTP, dCTP, and dUTP.

8. A kit for testing a novel coronavirus, the kit comprising a specimen treatment liquid containing sodium hydroxide as a main component, a reaction liquid, an internal standard substance, a primer, a probe, a reverse transcriptase, and a PCR enzyme.

9. The kit for testing a novel coronavirus according to claim 8, wherein the specimen treatment liquid, the reaction liquid, the internal standard substance, the primer, the probe, the reverse transcriptase, and the PCR enzyme are contained in two to four containers.

10. The kit for testing a novel coronavirus according to claim 9, wherein the specimen treatment liquid, the reaction liquid and the internal standard substance, the primer and the probe, and the reverse transcriptase and the PCR enzyme are each independently contained in four containers.

11. The kit for testing a novel coronavirus according to claim 8, wherein the specimen treatment liquid or the reaction liquid contains dATP, dGTP, dCTP and dTTP.

12. The kit for testing a novel coronavirus according to claim 8, wherein the specimen treatment liquid contains dATP, dGTP, dCTP, and dUTP.


SEQUENCE LISTING (12 sequences)

SEQ ID NO: 1 - DNA, 19 bp, Artificial Sequence
  Synthetic oligonucleotide to act as a PCR forward primer to amplify N1 region fragment.
cacattggcacccgcaatc

SEQ ID NO: 2 - DNA, 20 bp, Artificial Sequence
  Synthetic oligonucleotide to act as a PCR reverse primer to amplify N1 region fragment.
gaggaacgagaagaggcttg

SEQ ID NO: 3 - DNA, 20 bp, Artificial Sequence
  Synthetic oligonucleotide to act as a PCR forward primer to amplify N2 region fragment.
aaattttggggaccaggaac

SEQ ID NO: 4 - DNA, 20 bp, Artificial Sequence
  Synthetic oligonucleotide to act as a PCR reverse primer to amplify N2 region fragment.
tggcagctgtgtaggtcaac

SEQ ID NO: 5 - DNA, 20 bp, Artificial Sequence
  Synthetic oligonucleotide to act as a PCR forward primer to amplify N1 region fragment.
gaccccaaaatcagcgaaat

SEQ ID NO: 6 - DNA, 24 bp, Artificial Sequence
  Synthetic oligonucleotide to act as a PCR reverse primer to amplify N1 region fragment.
tctggttactgccagttgaatctg

SEQ ID NO: 7 - DNA, 20 bp, Artificial Sequence
  Synthetic oligonucleotide to act as a PCR forward primer to amplify N2 region fragment.
ttacaaacattggccgcaaa

SEQ ID NO: 8 - DNA, 18 bp, Artificial Sequence
  Synthetic oligonucleotide to act as a PCR reverse primer to amplify N2 region fragment.
gcgcgacattccgaagaa

SEQ ID NO: 9 - DNA, 25 bp, Artificial Sequence
  Synthetic oligonucleotide to act as a probe to detect amplified N1 region fragment.
acttcctcaaggaacaacattgcca

SEQ ID NO: 10 - DNA, 20 bp, Artificial Sequence
  Synthetic oligonucleotide to act as a probe to detect amplified N2 region fragment.
atgtcgcgcattggcatgga

SEQ ID NO: 11 - DNA, 24 bp, Artificial Sequence
  Synthetic oligonucleotide to act as a probe to detect amplified N1 region fragment.
accccgcattacgtttggtggacc

SEQ ID NO: 12 - DNA, 23 bp, Artificial Sequence
  Synthetic oligonucleotide to act as a probe to detect amplified N1 region fragment.
acaatttgcccccagcgcttcag